(19) United States
(12) Patent Application Publication
(10) Pub. No.: US 2024/0423566 A1
(43) Pub. Date: Dec. 26, 2024

(54) X-RAY DIAGNOSTIC APPARATUS AND CONTROL METHOD FOR X-RAY DIAGNOSTIC APPARATUS

(71) Applicant: CANON MEDICAL SYSTEMS CORPORATION, Otawara-shi (JP)

(72) Inventors: Ritsu YOKOHAMA, Nasushiobara (JP); Masashi HIRASAWA, Otawara (JP); Keisuke SUGAWARA, Otawara (JP); Hiroshi YOSHIDA, Utsunomiya (JP); Kazuo IMAGAWA, Nasushiobara (JP); Katsuaki SHINODA, Shioyagunshioyamachi (JP); Hisayasu YUMIZA, Otawara (JP)

(73) Assignee: CANON MEDICAL SYSTEMS CORPORATION, Otawara-shi (JP)

(21) Appl. No.: 18/742,090

(22) Filed: Jun. 13, 2024

(30) Foreign Application Priority Data
Jun. 20, 2023 (JP) 2023-100837

(51) Int. Cl. A61B 6/00; A61B 6/03; A61B 6/46; A61B 6/50

(52) U.S. Cl. CPC A61B 6/4452; A61B 6/03; A61B 6/4441; A61B 6/461; A61B 6/504

(57) ABSTRACT

In one embodiment, an X-ray diagnostic apparatus includes a memory and processing circuitry. The processing circuitry causes the memory to store a movement path that is defined by a combination of movements of a plurality of movable shafts of an imaging apparatus and is generated based on movements of the imaging apparatus in response to a user's operation. The processing circuitry moves the imaging apparatus in a reverse order of the movement path stored in the memory.

### CROSS-REFERENCE TO RELATED APPLICATION

This application is based upon and claims the benefit of priority from Japanese Patent Application No. 2023-100837, filed on Jun. 20, 2023, the entire contents of which are incorporated herein by reference.

### FIELD

Disclosed embodiments relate to an X-ray diagnostic apparatus and a control method for an X-ray diagnostic apparatus.

### BACKGROUND

An X-ray diagnostic apparatus is provided with a technique called automatic positioning or auto-positioning. The auto-positioning enables a C-arm or a bed to automatically move to an arbitrary pre-registered position by, for example, a rotational movement around each axis of the C-arm or a parallel movement along a ceiling rail while an input switch of the X-ray diagnostic apparatus is on.

When a user causes the C-arm to perform a desired movement by using the auto-positioning of the C-arm, it is necessary to register a desired movement position in advance, which increases effort during a medical procedure. In the case where the user manually operates the C-arm or the bed without registering the position before movement, if it is necessary to return the C-arm or the bed to the position before the movement, the user is required to perform complicated operations.

Although a movement from the current position to a designated position can be reproduced in the auto-positioning, there may be a plurality of paths for the C-arm to reproduce this movement. However, in the auto-positioning, the X-ray diagnostic apparatus does not acquire the movement path of the C-arm. Thus, it is almost impossible to uniquely reproduce the movement path of the C-arm that is manually and arbitrarily moved by a user such as a medical imaging technologist, and the C-arm may perform a movement that the user does not expect.

### BRIEF DESCRIPTION OF THE DRAWINGS

### In the Accompanying Drawings:

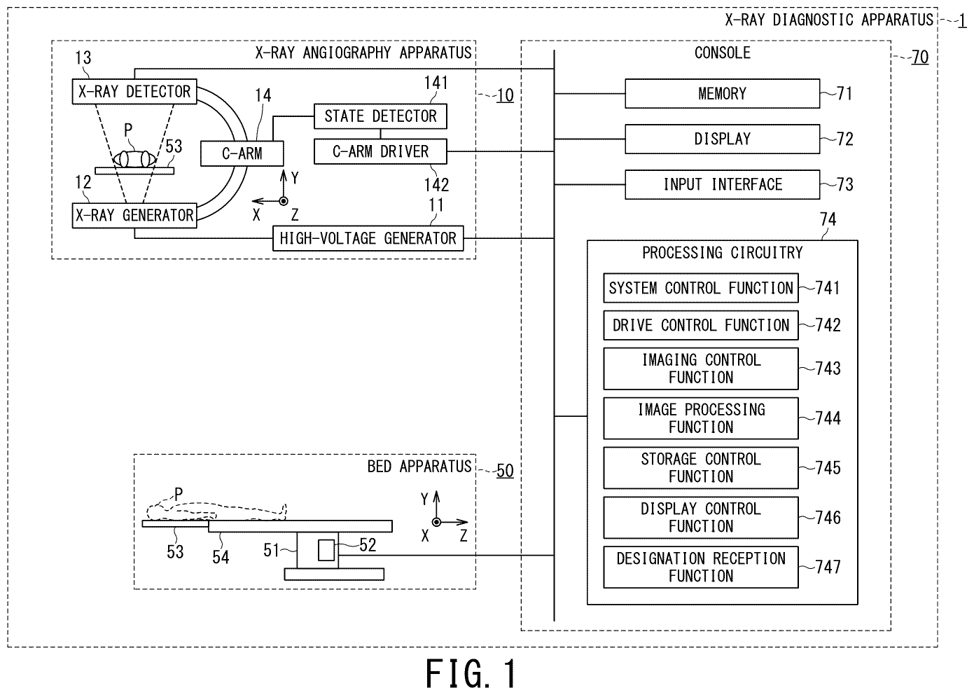

FIG. 1 is a block diagram illustrating a configuration of an X-ray diagnostic apparatus according to the first embodiment;

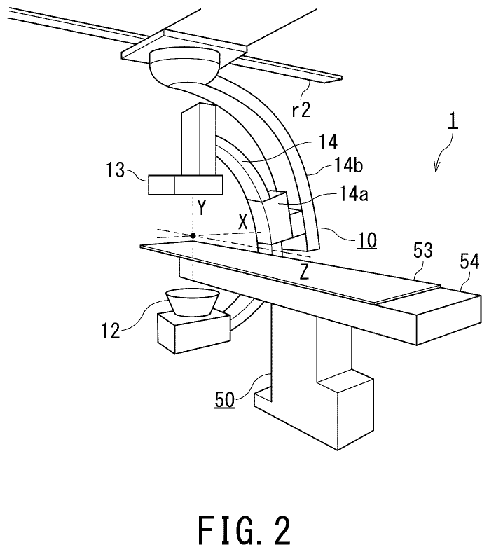

FIG. 2 is a perspective view illustrating an appearance of the X-ray diagnostic apparatus;

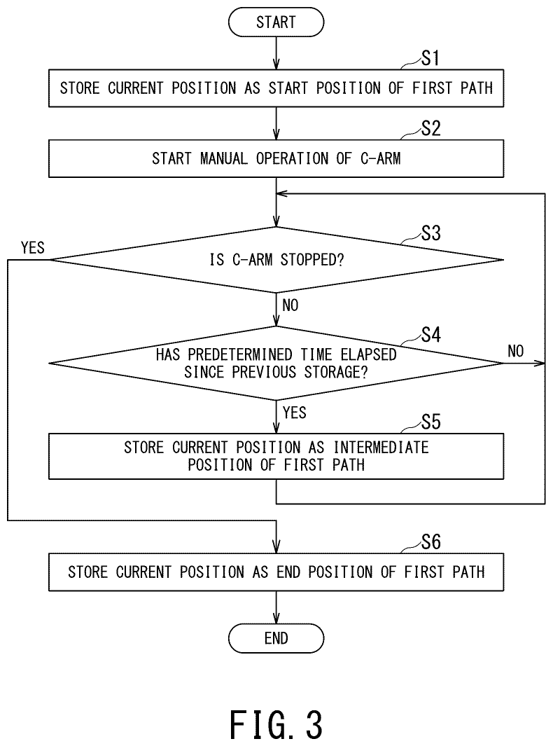

FIG. 3 is a flowchart illustrating processing of storing a movement path of an imaging apparatus to be manually moved according to the first embodiment:

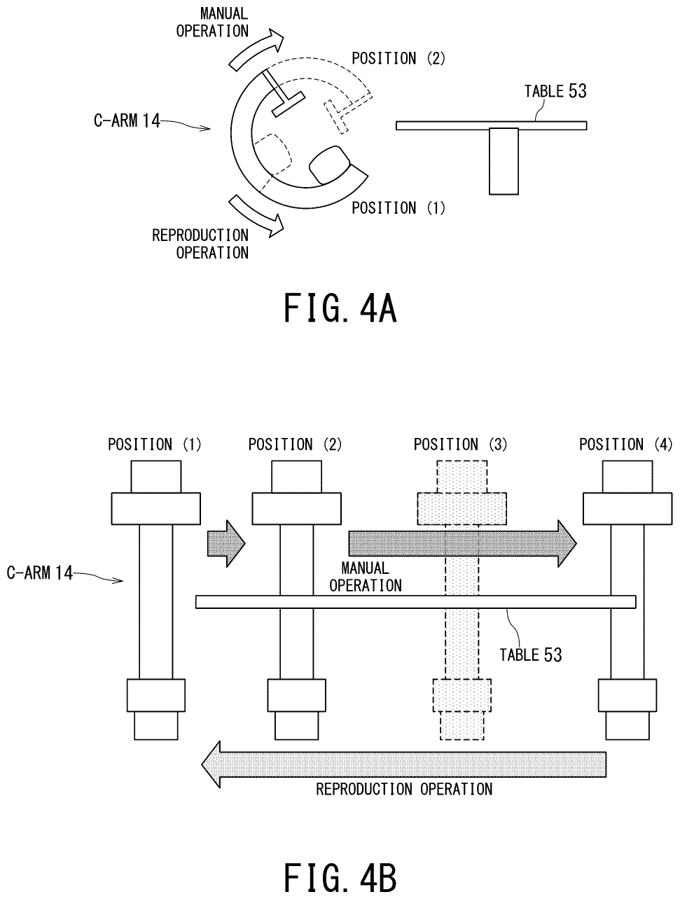

FIG. 4A is a schematic diagram illustrating a rotational movement of a C-arm according to the first embodiment;

FIG. 4B is a schematic diagram illustrating a parallel movement of the C-arm according to the first embodiment;

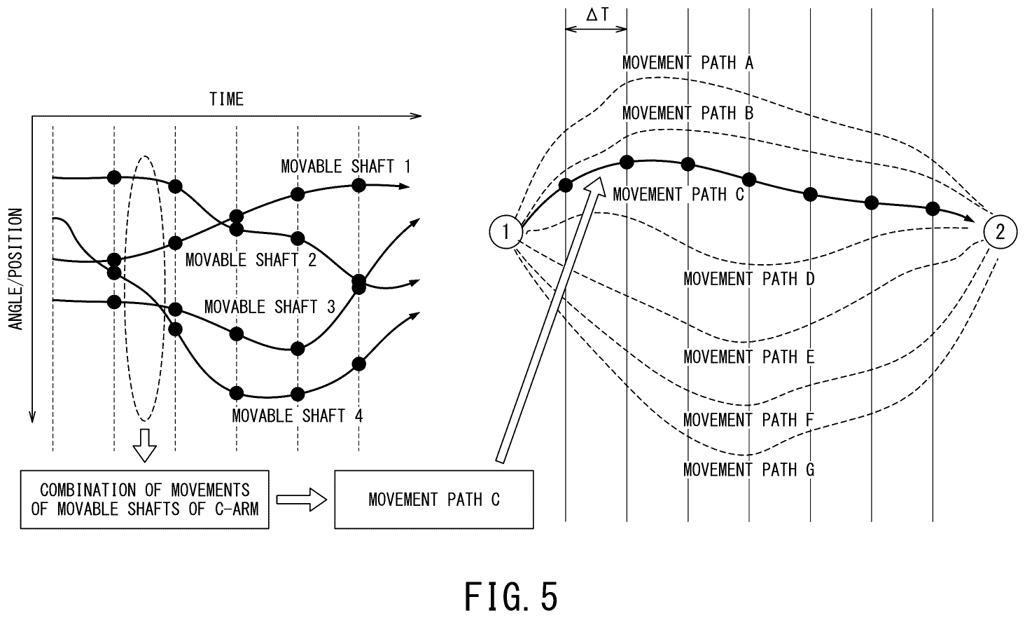

FIG. 5 is a schematic diagram illustrating a concept of a movement path according to the first embodiment;

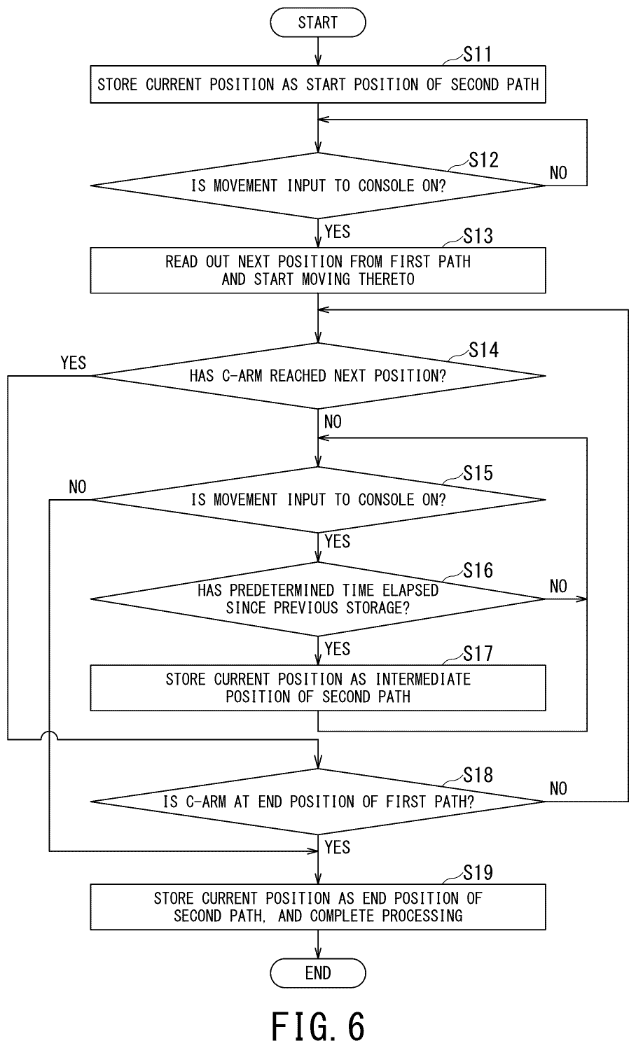

FIG. 6 is a flowchart illustrating reproduction-and-storage processing of the X-ray diagnostic apparatus according to the first embodiment;

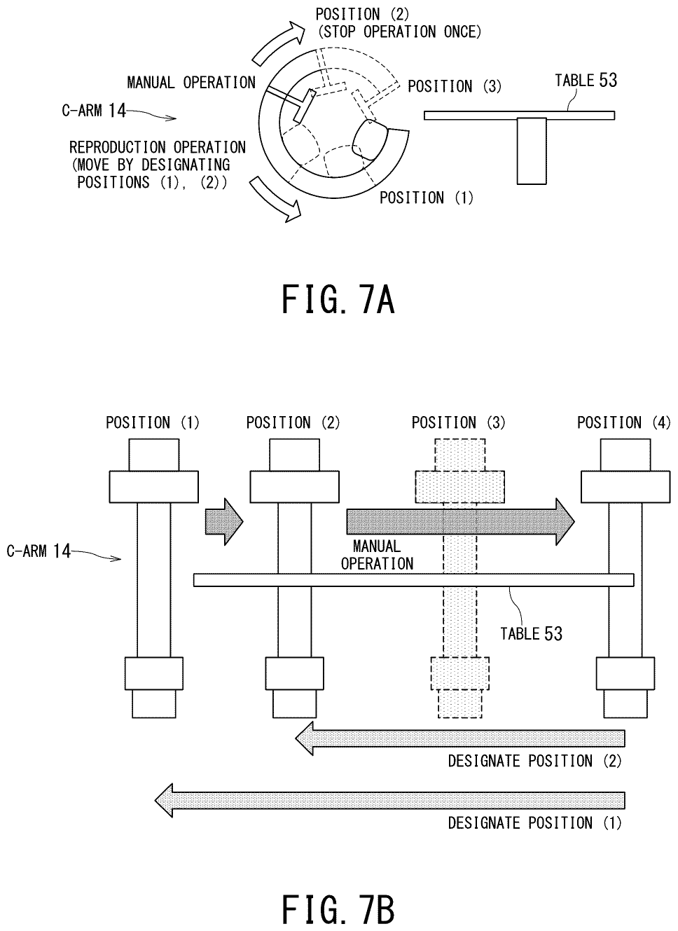

FIG. 7A is a schematic diagram illustrating the rotational movement of the C-arm according to the second embodiment;

FIG. 7B is a schematic diagram illustrating the parallel movement of the C-arm according to the second embodiment;

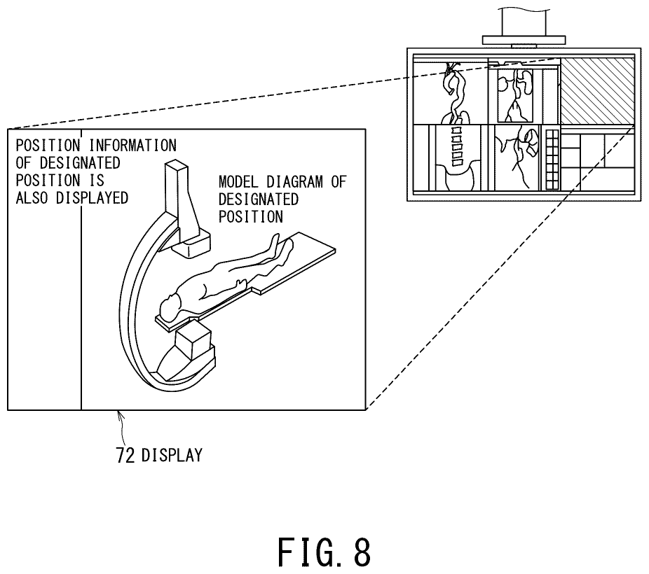

FIG. 8 is a schematic diagram illustrating a model diagram according to the second embodiment;

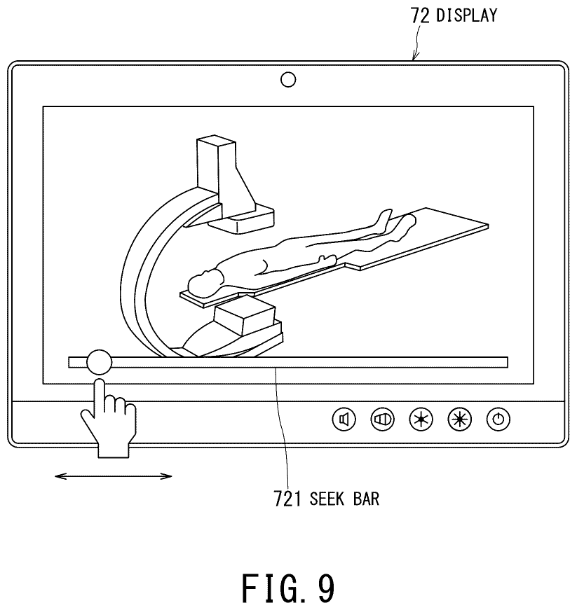

FIG. 9 is a schematic diagram illustrating a model diagram according to the third embodiment;

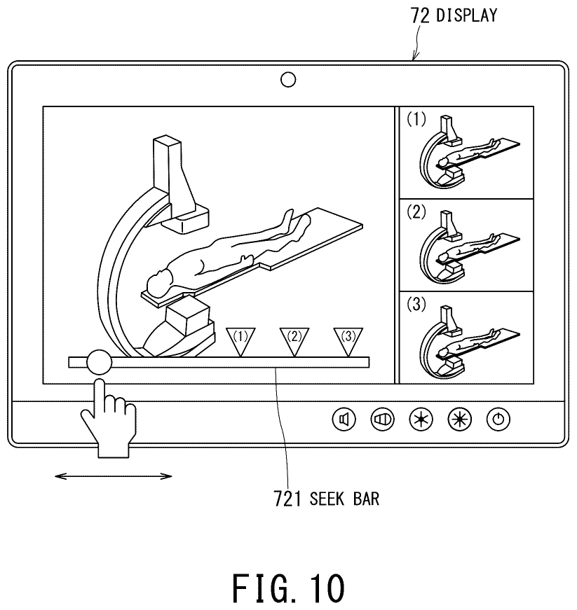

FIG. 10 is a schematic diagram illustrating another model diagram according to the third embodiment;

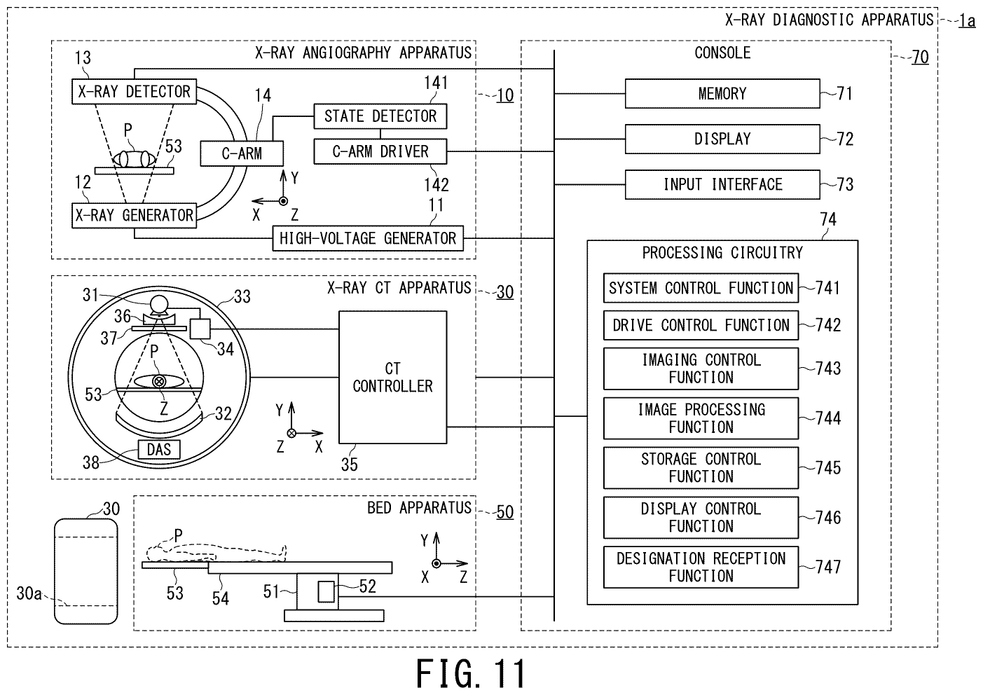

FIG. 11 is a block diagram illustrating a configuration of the X-ray diagnostic apparatus according to the fourth embodiment;

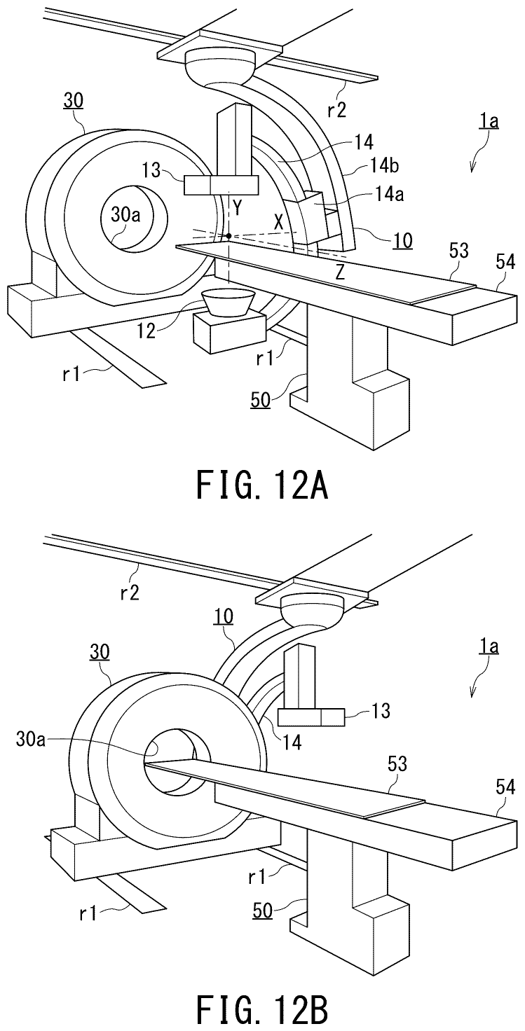

FIG. 12A is a perspective view illustrating an appearance of the X-ray diagnostic apparatus; and

FIG. 12B is another perspective view illustrating the appearance of the X-ray diagnostic apparatus.

### DETAILED DESCRIPTION

Hereinbelow, embodiments of an X-ray diagnostic apparatus and a control method for an X-ray diagnostic apparatus will be described in detail by referring to the accompanying drawings.

In one embodiment, an X-ray diagnostic apparatus includes a memory and processing circuitry. The processing circuitry causes the memory to store a movement path that is defined by a combination of movements of a plurality of movable shafts of an imaging apparatus and is generated based on movements of the imaging apparatus in response to a user's operation. The processing circuitry moves the imaging apparatus in a reverse order of the movement path stored in the memory.

### First Embodiment

FIG. 1 is a block diagram illustrating a configuration of an X-ray diagnostic apparatus 1 according to the first embodiment. FIG. 2 is a perspective view illustrating an appearance of the X-ray diagnostic apparatus 1. The X-ray diagnostic apparatus 1 includes an X-ray angiography apparatus 10, a bed apparatus 50, and a console 70.

The X-ray angiography apparatus 10 includes a high-voltage generator 11, an X-ray generator 12, an X-ray detector 13, a C-arm 14, a state detector 141, and a C-arm driver 142.

The high-voltage generator 11 generates and outputs a high voltage to be applied between an anode and a cathode of an X-ray tube in order to accelerate thermoelectrons generated from the cathode. The X-ray generator 12 includes: an X-ray tube configured to irradiate an object P with X-rays; and an X-ray aperture and ROI (Region Of Interest) filter having a function to attenuate or reduce X-ray irradiation dose.

The X-ray tube generates X-rays. Specifically, the X-ray tube is a vacuum tube that holds both the cathode configured to generate thermoelectrons and the anode configured to receive thermoelectrons from the cathode and thereby generate X-rays. The X-ray tube is connected to the high-voltage generator 11 via a high-voltage cable. A tube voltage is applied between the cathode and the anode by the high-voltage generator 11. Application of the tube voltage causes the thermoelectrons to move from the cathode toward the anode, which generates a flow of a tube current. Supply of the filament current and application of the high voltage from the high-voltage generator 11 cause the thermoelectrons to move from the cathode toward the anode, and X-rays are generated by having the thermoelectrons collide with the anode.

The ROI filter is located between the X-ray tube and the X-ray aperture, and is composed of a metal plate such as copper and aluminum. The ROI filter has an opened region at least in part, for example in the center, and attenuates X-rays outside the opened region. Thus, the ROI filter causes all the X-rays having passed the opened region to fully pass through, and causes the X-rays having passed the other regions to attenuate and then transmit therethrough.

The X-ray aperture is located between the X-ray tube and the X-ray detector 13, and is composed of a lead plate as a metal plate. The X-ray aperture narrows down X-rays generated by the X-ray tube in such a manner that X-rays are radiated only to the region of interest of the object P by blocking X-rays outside the opened region.

The X-ray detector 13 detects X-rays that have passed through the object P, and two configurations of such an X-ray detector 13 are available. One of them is configured to directly convert X-rays into electric charge, and the other is configured to convert X-rays into light and then convert the light into electric charge. Although the former will be described in the following, the X-ray detector 13 may be configured as the latter one. That is, the X-ray detector 13 includes: a planar FPD (Flat Panel Detector) configured to convert X-rays having passed through the object P into electric charge and accumulate the electric charge; and a gate driver configured to generate a drive pulse for reading out the electric charge accumulated in the FPD, for example. The accumulated electric charge is sequentially read out by the drive pulse supplied by the gate driver.

In the output stage of the X-ray detector 13, a projection data generation circuit and a projection data storage circuit (not shown) are provided. The projection data generation circuit converts a digitally converted parallel signal into a time-sequential serial signal, and then supplies the serial signal as time-sequential projection data to the projection data storage circuit. The projection data storage circuit generates two-dimensional projection data by sequentially storing the time-sequential projection data supplied from the projection data generation circuit. These two-dimensional projection data are stored in a memory 71 described below.

The C-arm 14 has a configuration of enabling X-ray imaging of the object P on the table 53 by holding and having the X-ray generator 12 and the X-ray detector 13 face each other with the object P and the table 53 interposed therebetween. The C-arm 14 is one example of an imaging apparatus. In FIG. 2, the C-arm 14 is close to the table 53.

The C-arm 14 is held by a holding unit 14a so as to be rotatable around the axis in the X direction that is perpendicular to both the Y direction perpendicular to the table 53 and the Z direction along the longitudinal axis direction of the table 53. Further, the C-arm 14 has a substantially arc shape centered on the axis in the Z direction, and is held by the holding unit 14a so as to be slidable along the substantially arc shape. In other words, the C-arm 14 can perform a sliding movement around the axis in the Z direction.

Further, the C-arm 14 can perform a rotational movement centered on the holding unit 14a, i.e., a rotational movement around the axis in the X direction. The combination of the sliding movement and the rotational movement enables X-ray imaging from various angles. Since the C-arm 14 can rotate together with a support arm 14b around the axis in the Y direction, the rotation center axis of the above-described sliding movement can be set in the X direction, for example.

Returning to FIG. 1, a plurality of power sources for performing movements related to the X-direction axis, Y-direction axis, Z-direction axis, and the support arm 14b under a rail r2 are provided in appropriate positions of the C-arm 14. These power sources constitute the C-arm driver 142. The C-arm driver 142 reads in the drive signal from a drive control function 742 described below, and causes the C-arm 14 to perform a sliding movement, a rotational movement, and a rectilinear movement. Furthermore, the C-arm 14 is provided with the state detector 141 that detects its angle, attitude, and position. The state detector 141 is composed of a potentiometer configured to detect the rotation angle and/or movement amount of the C-arm 14 and an encoder configured as a position detection sensor, for example.

The bed apparatus 50 is an apparatus for placing the object P thereon and moving the object P, and includes a base 51, a bed driver 52, the table 53, and a support frame 54.

The base 51 is installed on the floor surface, and is a housing configured to support the support frame 54 in such a manner that the support frame 54 can move in the vertical direction (i.e., in the Y direction).

The bed driver 52 is housed in a housing of the bed apparatus 50, and includes a motor or actuator configured to move the table 53 with the object P placed thereon in the longitudinal direction (i.e., in the Z direction) of the table 53. The bed driver 52 reads in the drive signal from the drive control function 742 so as to move the table 53 horizontally and/or vertically with respect to the floor surface. Similarly, the bed driver 52 reads in the drive signal from the drive control function 742 so as to tilt the table 53 around the short axis of the table 53. The bed driver 52 may tilt the table 53 by tilting the support frame 54. The positional relationship between the imaging axis and the object P is changed by moving the C-arm 14 or the table 53. In addition to moving the table 53, the bed driver 52 may also move the support frame 54 in the longitudinal direction of the table 53.

The table 53 is provided on the upper surface of the support frame 54, and is a plate on which the object P is placed.

The support frame 54 movably supports the table 53 on which the object P is placed. In detail, the support frame 54 is provided on the upper portion of the base 51, and supports the table 53 in a slidable manner along its longitudinal direction.

The console 70 includes a memory 71, a display 72, an input interface 73, and processing circuitry 74.

The memory 71 includes: a memory body configured to store electrical information, such as a hard disk drive (HDD); and peripheral circuits such as a memory controller and a memory interface that are attached to or belong to the memory body. For example, the memory 71 stores: programs to be executed by the processing circuitry 74; detection data received from a DAS (Data Acquisition System) 38; medical images generated by the processing circuitry 74; data to be used for processing by the processing circuitry 74; various table data; data during processing; and data after processing. The medical images include a CT image, a 3D-CT image, a 3D angiography image, a 2D angiography image, a superimposed image, and a processed image, for example.

The display 72 displays various information items such as medical images. The display 72 outputs medical images generated by the processing circuitry 74 and a GUI (Graphical User Interface) for receiving various operations from a user, for example. The display 72 is a liquid crystal display or a CRT (Cathode Ray Tube) display, for example. Further, the display 72 may be provided in an X-ray CT apparatus 30, which is described below as another example of the imaging apparatus in the fourth embodiment. In addition, the display 72 may be of a desktop type or may be composed of a tablet terminal that can wirelessly communicate with the main body of the console 70, for example.

The input interface 73 is used for entering object information, setting X-ray conditions, and inputting various command signals, for example. The object information includes an object ID, an object name, date of birth, age, height, weight, gender, and an examination site, for example. The input interface 73 is realized by components for instructing the movement of the C-arm 14 and setting a region of interest (ROI), such as a trackball, a switch button, a mouse, a keyboard, a touchpad allowing the user to perform input operations by touching an operation screen, and a touch panel display in which a display screen and a touchpad are integrated. The input interface 73 is connected to the processing circuitry 74, converts the input operation received from the user into an electrical signal, and outputs the electrical signal to the processing circuitry 74. Note that the input interface 73 may be provided in the X-ray CT apparatus 30. Furthermore, the input interface 73 may be configured as a tablet terminal that can wirelessly communicate with the main body of the console 70, for example.

The processing circuitry 74 is a processor that reads in and executes programs from the memory 71 so as to implement a system control function 741, the drive control function 742, an imaging control function 743, an image processing function 744, a storage control function 745, a display control function 746, and a designation reception function 747 corresponding to the respective programs. The processing circuitry 74 may be configured by combining a plurality of independent processors, each of which implements each function by executing the program.

The system control function 741 includes a function to: temporarily store information such as a command signal inputted via the input interface 73 by the user and various initial setting conditions; and then transmit these information items to each processing function of the processing circuitry 74, for example.

The drive control function 742 includes a function to use the information on the driving of the X-ray CT apparatus 30, the C-arm 14, and the table 53 inputted via the input interface 73 for controlling the C-arm driver 142, the bed driver 52, and a CT controller 35, which is described below in the fourth embodiment as a main controller of the X-ray CT apparatus 30, for example. The drive control function 742 includes a function to move the imaging apparatus in the reverse order of the movement path stored in the memory 71, for example. The movement path is defined by a combination of movements of a plurality of movable shafts of the imaging apparatus, and is generated based on the actual movement of the imaging apparatus in response to a user's operation.

Aspects of the imaging apparatus include the C-arm 14, the X-ray CT apparatus 30, and the table 53. The plurality of movable shafts of the imaging apparatus include the plurality of movable shafts of the C-arm 14 of the X-ray angiography apparatus 10 and the plurality of movable shafts of the table 53. The plurality of movable shafts of the imaging apparatus further include a movement shaft of the X-ray CT apparatus 30 with respect to the table 53, wherein the X-ray CT apparatus 30 is to be moved in combination with the X-ray angiography apparatus 10. The table 53 is an example of a bed. Furthermore, the drive control function 742 includes a function to move the imaging apparatus in such a manner that a forward-direction movement path is reproduced in the reverse direction.

The drive control function 742 controls: the parallel movement and/or the rotational movement of the X-ray angiography apparatus 10; the parallel movement, the rotational movement, and the tilt of the X-ray CT apparatus 30; and the parallel movement and the tilt of the bed apparatus 50, for example. In addition to tilting the table 53, the drive control function 742 may also change the height of the table 53. The tilt and change of the height of the table 53 may be performed in parallel or in series. In addition, the drive control function 742 performs interference control so as to avoid contact between the X-ray angiography apparatus 10 and the object P on the basis of an interference region having been set.

The imaging control function 743 includes a function to read in information from the system control function 741 and control X-ray conditions such as an irradiation time, a tube current, and a tube voltage in the high-voltage generator 11, for example. The X-ray conditions may include a product of a tube current and an irradiation time (mAs).

As to X-ray CT imaging, the image processing function 744 includes a function to generate data obtained by subjecting detection data in the memory 71 to pre-processing such as logarithmic conversion processing, offset correction processing, sensitivity correction processing between channels, and beam hardening correction, for example.

The storage control function 745 includes a function to cause the memory 71 to store a movement path, which is defined by a combination of movements of the plurality of movable shafts of the imaging apparatus and is generated by the imaging apparatus moving in response to a user's operation. The user's operation may be a manual operation in which the user manually sets the plurality of movable shafts of the imaging apparatus. The user's operation may be performed between a plurality of movements of the imaging apparatus in response to an instruction of the auto-positioning. The user's operation may be an operation related to the auto-positioning that reads out and executes pre-registered motion settings of the movable shafts of the imaging apparatus.

The display control function 746 includes a function to read in signals from the system control function 741, acquire desired medical image data from the memory 71, and display them on the display 72, for example. The display control function 746 also includes a function to display a pseudo image of the imaging apparatus corresponding to respective positions on the movement path stored in the memory 71 as a moving image on the display 72.

The designation reception function 747 includes a function to receive one position, which is on the movement path of the imaging apparatus stored in the memory 71 and is designated by the user, as a stop position of the imaging apparatus.

The processing to be performed by the X-ray diagnostic apparatus 1 according to the first embodiment is roughly classified into two patterns including: processing of storing the movement path of the imaging apparatus to be manually moved; and processing of moving the imaging apparatus while reproducing the stored movement path under the control of the X-ray diagnostic apparatus 1.

Of the above-described two patterns of processing, the processing of storing the movement path of the imaging apparatus to be manually moved is illustrated as a flowchart of FIG. 3.

FIG. 4A illustrates the rotational movement of the C-arm 14 according to the first embodiment, and FIG. 4B illustrates the parallel movement of the C-arm 14 according to the first embodiment.

FIG. 5 illustrates a concept of the movement path according to the first embodiment. In particular, FIG. 5 is a schematic diagram for illustrating the movement path defined by a combination of movements of the plurality of movable shafts of the imaging apparatus.

This storage processing is processing of storing the movement path of the imaging apparatus in accordance with the user's manual operations. This storage processing is mainly executed by the processing circuitry 74 of the console 70. Note that the manual operations by the user are performed except each movement period of the imaging apparatus under the auto-positioning in which the position and driving details of the imaging apparatus are determined in advance. In other words, this storage processing is applied to a case where the user freely moves the imaging apparatus between respective auto-positioning operations and freely changes settings through input operations. In the following, a description will be given of the case where the imaging apparatus is the C-arm 14. Although a ceiling-suspended C-arm 14 is described below as one example, this storage processing can be applied to a floor-mounted C-arm and a ceiling-suspended Q-arm in a similar manner.

In the step S1, the storage control function 745 of the processing circuitry 74 causes the memory 71 to store the current position of the C-arm 14 (i.e., the operation start position) as the start position of the first path. The current position is a combination of angles and positions of the respective movable shafts of the C-arm 14, and is stored in the memory 71 in a stack format with a FILO (First In Last Out) structure, for example.

In the step S2, the user starts a manual operation of the C-arm 14 of the X-ray angiography apparatus 10. This causes the C-arm 14 to start the rotational movement or the parallel movement. For example, as shown in FIG. 4A, the C-arm 14 may perform the rotational movement from the position (1) to the position (2). As shown in FIG. 4B, the C-arm 14 may perform the parallel movement from the position (1) to the position (4) via the positions (2) and (3). In addition, the C-arm 14 may perform a combination movement of the rotational movement and the parallel movement.

In the step S3, the drive control function 742 of the processing circuitry 74 determines whether the C-arm 14 is stopped or not. For example, the drive control function 742 acquires detected values of the rotation angle, movement amount, and position of the C-arm 14 from the state detector 141 of the X-ray angiography apparatus 10, and performs the above-described determination on the basis of those detected values. If the C-arm 14 is stopped (YES in the step S3), the processing circuitry 74 advances the processing to the step S6. If the C-arm 14 is not stopped (NO in the step S3), the processing circuitry 74 advances the processing to the step S4.

In the step S4, the processing circuitry 74 determines whether a predetermined time has elapsed from a time point of storing the position of the C-arm 14 or not. This predetermined time is a relatively short time length (for example, 1 to 2 seconds) during which the movement path of the C-arm 14 can be uniquely reproduced between the previous and subsequent positions. If the predetermined time has elapsed (YES in the step S4), the processing circuitry 74 advances the processing to the step S5. If the predetermined time has not elapsed (NO in the step S4), the processing circuitry 74 returns the processing to the step S3.

In the step S5, the storage control function 745 of the processing circuitry 74 causes the memory 71 to store the current position of the C-arm 14 as an intermediate position of the first path (for example, (1)-1, (1)-2, (1)-3, . . . ). In other words, when the C-arm 14 is moving in accordance with the user's manual operation as shown in the left part of FIG. 5, the storage control function 745 causes the memory 71 to store the combination of movements of the plurality of movable shafts of the C-arm 14 as a movement path at predetermined time intervals. The movement of each movable shaft refers to change in the rotation angle and/or the movement position of each of the movable shafts 1 to 4 before and after the predetermined time, for example. The movement path of the C-arm 14 can be uniquely identified by storing the position of the C-arm 14 at predetermined time intervals.

Conversely, if only the position (2) is stored after elapse of the predetermined time or longer from the time point of the position (1) of the C-arm 14, the movement path between the position (1) and the position (2) cannot be uniquely identified. As shown in the right part of FIG. 5, for example, storing only the start position (1) and the end position (2) when moving the C-arm 14 does not enable identification of the actual movement path C, and a movement on the movement path A, B, D, E, F, or G are possible paths. For this reason, as shown by the movement path C in FIG. 5, the storage control function 745 stores the position of the C-arm 14 at predetermined time intervals (for example, ΔT). Afterward, the processing circuitry 74 returns the processing to the step S3.

In the step S6, the storage control function 745 of the processing circuitry 74 causes the memory 71 to store the current position (i.e., operation stop position) of the C-arm 14 as the end position of the first path.

As shown in FIG. 4A, if the C-arm 14 makes the rotational movement from the position (1) to the position (2) in a period shorter than the predetermined time, in the storage processing of FIG. 3, after repeating the steps S3 and S4, the processing proceeds to the step S6 without proceeding to the step S5. Additionally, in the case of adding a movement to an arbitrary position such as the position (3) and the position (4) after the position (2), similar storage processing from the position (1) to the position (2) is performed.

FIG. 6 is a flowchart illustrating reproduction-and-storage processing of the X-ray diagnostic apparatus 1 according to the first embodiment. This reproduction-and-storage processing is processing of moving the imaging apparatus in the reverse order of the stored movement path and storing this reverse movement path. This reproduction-and-storage processing is mainly executed by the processing circuitry 74 of the console 70. In the following, a description will be given of the case where the imaging apparatus is the C-arm 14 as one example.

To summarize, when the movement path of the C-arm 14 stored by the storage processing in FIG. 3 is defined as the first path, the drive control function 742 moves the C-arm 14 in the reverse order of the first path. While the drive control function 742 is moving the C-arm 14 in the reverse order of the first path, the storage control function 745 causes the memory 71 to further store this reverse movement path as the second path. This allows the drive control function 742 to further move the C-arm 14 in the reverse order of the second path. The processing in FIG. 6 will be described in detail below.

In the step S11, the storage control function 745 of the processing circuitry 74 causes the memory 71 to store the current position of the C-arm 14 as the start position of the second path (i.e., as the reproduction start position).

In the step S12, it is determined whether a movement input by the user via the input interface 73 of the console 70 is on or not. The input interface 73 for receiving an input of the start of movement includes a switch type and a lever type, for example. In the switch type, while the user input is on, the drive control function 742 causes the C-arm driver 142 to perform a reproduction movement of the C-arm 14 at a predetermined speed. In the lever type, while the user input is on, the drive control function 742 causes the C-arm driver 142 to perform the reproduction movement of the C-arm 14 at a speed in accordance with an inclination angle of the lever under the condition that a predetermined speed is the upper speed limit. The predetermined speed is, for example, the movement speed of the C-arm 14 during normal manual operation. In other words, the drive control function 742 moves the C-arm 14 in the reverse order when the instruction to move the C-arm 14 in the reverse direction continues by the user's operation.

If the movement input to the input interface 73 is on (YES in the step S12), the processing circuitry 74 advances the processing to the step S13. If the movement input to the input interface 73 is not on (NO in the step S12), the processing circuitry 74 repeats processing of the step S12.

In the step S13, the drive control function 742 of the processing circuitry 74 reads out the next position based on the first path stored in the memory 71, and causes the C-arm driver 142 to start moving the C-arm 14 to the next position. Each position of the first path is stored in the memory 71 in the form of a FILO-structured stack, for example, so it is read out in the order of being most recently stored.

As a result, the C-arm 14 starts the rotational movement or the parallel movement. For example, as shown in FIG. 4A, the C-arm 14 may perform the rotational movement from the position (2) to the position (1). As shown in FIG. 4B, the C-arm 14 may perform the parallel movement from the position (4) to the position (1) via the positions (3) and (2). In addition, the C-arm 14 may perform a complex movement in which the rotational movement and the parallel movement are combined by sequentially changing the movable shafts.

While the user input is on, the drive control function 742 causes the C-arm 14 to perform the reproduction movement as long as the next position data are stored in the memory 71. When the user releases the switch or the lever, the user input is turned off and the C-arm 14 stops.

In the step S14, the drive control function 742 of the processing circuitry 74 determines whether the C-arm 14 has reached the next position or not. If the C-arm 14 has reached the next position (YES in the step S14), the processing circuitry 74 advances the processing to the step S18. If the C-arm 14 has not yet reached the next position (NO in the step S14), the processing circuitry 74 advances the processing to the step S15.

In the step S15, it is determined whether the movement input to the input interface 73 of the console 70 by the user is on or not. If the movement input to the input interface 73 by the user is on (YES in the step S15), the processing circuitry 74 advances the processing to the step S16. If the movement input to the input interface 73 by the user is not on (NO in the step S15), the processing circuitry 74 advances the processing to the step S19.

In the step S16, the processing circuitry 74 determines whether a predetermined time has elapsed from the time point at which the position of the C-arm 14 is stored or not. If the predetermined time has elapsed (YES in the step S16), the processing circuitry 74 advances the processing to the step S17. If the predetermined time has not yet elapsed (NO in the step S16), the processing circuitry 74 returns the processing to the step S15.

In the step S17, the storage control function 745 of the processing circuitry 74 causes the memory 71 to store the current position of the C-arm 14 as the intermediate position of the second path, and then the processing circuitry 74 returns the processing to the step S15.

In the step S18, the drive control function 742 of the processing circuitry 74 determines whether the C-arm 14 has reached the end position of the first path or not. If the C-arm 14 reaches the end position of the first path (YES in the step S18), the processing circuitry 74 advances the processing to the step S19. If the C-arm 14 does not reach the end position of the first path (NO in the step S18), the processing circuitry 74 returns the processing to the step S14.

In the step S19, the storage control function 745 of the processing circuitry 74 causes the memory 71 to store the current position of the C-arm 14 as the end position of the second path (i.e., the reproduction end position), and then the processing circuitry 74 completes the series of processing.

Note that each position in the movement path is overwritten depending on the capacity of the memory region of the memory 71 for storing the movement path. Storing not only the first path but also the most recent second path can meet the needs of: returning the C-arm 14 to the immediately preceding position (i.e., the position right before the current position); and further returning the C-arm 14 to the position just one before the immediately preceding position. Since the C-arm 14 may be stopped midway during reproduction and the first and second paths are not necessarily the same, there is an advantage in storing the second path.

According to the above-described method, while the user input is on, the C-arm 14 continuously moves from the latest stored position of the first path to the immediately preceding stored position so as to move to the oldest stored position. This method enables reproduction of the first path. In addition, the user can have the C-arm 14 automatically move to the desired position without going through the trouble of storing the movement path of the C-arm 14. Reproducing the movement path of the C-arm 14 improves usability and enables automation of complicated operations.

The forward direction of the first path can be reproduced by holding down another switch at an arbitrary stop position of the C-arm 14 when moving in the reverse order of the first path (or by operating the switch to change settings or by having the lever switch to the opposite direction, for example). For example, in FIG. 4B, the C-arm 14 can move to the position (4) again after stopping at any position between the position (4) and the position (1).

### Second Embodiment

In the reproduction processing according to the second embodiment, when the user designates the stored position where the C-arm 14 is stopped as the end position, the drive control function 742 reproduces the movement of the C-arm 14 in such a manner that this designated position becomes the end position. In other words, the designation reception function 747 receives one position, which is on the movement path of the C-arm 14 stored in the memory 71 through the storage processing according to the first embodiment and is a position designated by the user, as the stop position of the C-arm 14. Further, the drive control function 742 automatically moves the C-arm 14 from an arbitrary position on the movement path of the C-arm 14 to the stop position in response to the start instruction from the user. To designate the stop position, for example, a numerical value indicating the order of the positions is inputted.

FIG. 7A illustrates the rotational movement of the C-arm 14 according to the second embodiment, and FIG. 7B illustrates the parallel movement of the C-arm 14 according to the second embodiment.

The conditions for performing the storage processing include: storing the positions (e.g., position (1), position (2), . . . ) corresponding to the stopped state of the C-arm 14; and storing the positions corresponding to the movement time of the C-arm 14 (e.g., position (1)-1, position (1)-2, . . . , position (2)-1, position (2)-2, . . . ). At this time, in the case of storing positions in the stopped state, the order is stored as 1, 2, 3, . . . starting from the oldest stored position. The order is updated every time the C-arm 14 moves. Designating the order enables setting of the end position of the C-arm 14.

For example, as shown in FIG. 7A, when the C-arm 14 is at the position (3), the position (1) or (2) can be designated as the stop position. In addition, as shown in FIG. 7B, when the C-arm 14 is at the position (4), the position (1) or (2) can be designated as the stop position. Note that the position (3) of the C-arm 14 in FIG. 7B is stored as a position corresponding to the movement time and thus cannot be designated as the stop position.

The reproduction processing in which the stop position is designated is performed as follows. The user inputs a numerical value to the input interface 73 of the console 70 (for example, designating the position (1) by inputting “1”), and then performs a movement start input. This movement start input causes the C-arm 14 to start moving. The C-arm 14 moves only while the user input is on, and automatically stops when reaching the designated position. Instead of using the input interface 73, the user may enter the numerical value or performs the movement start input operation on a tablet terminal and/or smartphone that can communicate with the console 70.

Since it is a timing after the manual operation on the C-arm 14 by the user, it is assumed that the user is aware of at least the start position (1) and the end position (2) of the movement path. The intermediate positions of the movement path (e.g., (1)-1, (1)-2) are stored at predetermined time intervals, so it is conceivable that the user intuitively knows the position of the C-arm 14 corresponding to the order. However, it is assumed that the user may have difficulty knowing the exact intermediate position of the C-arm 14.

FIG. 8 is a schematic diagram illustrating a model diagram according to the second embodiment. At the same time as a numerical value indicating the position order is entered into the console 70, the display control function 746 may cause the display 72 to display a model diagram indicative of the position of the C-arm 14 corresponding to this numerical value, as shown in FIG. 8.

In this manner, the model diagram indicative of the position of the C-arm 14 corresponding to the inputted value is displayed, so the user can input the start of movement after checking the actual stop position, rather than by intuition. Instead of the display 72, the display control function 746 may display the model diagram on a tablet terminal and/or smartphone that can communicate with the console 70.

### Third Embodiment

The X-ray diagnostic apparatus 1 according to the third embodiment displays the position of the C-arm 14 stored in the storage processing according to the first embodiment on a model diagram that is a continuous moving image (i.e., continuous animation). The X-ray diagnostic apparatus 1 can change the display of the moving image by using a seek bar 721, and displays the model diagram of an arbitrary position on the display 72. In this state, in response to the movement start input via console 70, the X-ray diagnostic apparatus 1 reproduces the movement of the C-arm 14 in such a manner that the position of the moving image becomes the end position.

In other words, the display control function 746 of the processing circuitry 74 causes the display 72 to further display the seek bar 721 that allows the user to designate a position corresponding to an arbitrary frame of the moving image as the stop position of the C-arm 14. The drive control function 742 moves the C-arm 14 until the C-arm 14 reaches the designated stop position. The position which the user can designate as the stop position has to be a position stored as the movement path. Accordingly, the user can designate an intermediate position (e.g., (1)-1, (1)-2, . . . ) of the movement path, but the user cannot designate a position between the position (1)-1 and the position (1)-2, for example.

FIG. 9 is a schematic diagram illustrating a model diagram according to the third embodiment. As shown in FIG. 9, when the user operates the seek bar 721 displayed at the lower part of the display 72 from side to side, the display of the moving image of the C-arm 14 on the display 72 is changed. When the user has the display 72 to display the desired position shown in the moving image, the desired position is set as the end position of the C-arm 14 in the reproduction movement.

At this time, the user can select one of the following two as the movement path of the C-arm 14.

### (1) Movement-Path Reproduction Route

As shown in the first embodiment, the X-ray diagnostic apparatus 1 moves the C-arm 14 so as to reproduce the movement path by causing the C-arm 14 to sequentially pass through the movement path stored in the memory 71.

### (2) Shortest-Distance Movement Route

The X-ray diagnostic apparatus 1 moves the C-arm 14 along the shortest-distance route from the current position of the C-arm 14 to the target position. In other words, in accordance with the user's selection, the drive control function 742 moves the C-arm 14 from the current position to the target position along the shortest path, instead of the movement path stored in the memory 71. For example, when the movement path is a route in which the C-arm 14 moves a distance L southward from the current position and then moves the same distance L eastward to reach the target position, the shortest route is the route in which the C-arm 14 moves southeastward from the current position to the target position directly.

This method enables to return the C-arm 14 to a specific position regardless of the route. In addition, the shortest-distance movement route is a route that the C-arm 14 has never passed before, but it can be selected as the shortest-time route if the safety is ensured.

FIG. 10 is a schematic diagram illustrating another model diagram according to the third embodiment. In addition to the display aspect shown in FIG. 9, the display control function 746 may automatically display a specific position on the moving image when the C-arm 14 is stopped for a predetermined time or longer ((1) in FIG. 10), when an X-ray irradiation switch is turned on ((2) in FIG. 10), or when the C-arm 14 moves for a predetermined time or longer at a designated speed or slower ((3) in FIG. 10). Each position may be displayed in a distinguishable manner by color or shape.

As shown in FIG. 10, the display control function 746 displays three points on the seek bar 721, for example. At that time, when the user selects any point, the display control function 746 causes the display 72 to display the specific image of the position corresponding to the selected point from the moving image.

In other words, the storage control function 745 causes the memory 71 to store the specific positions on the movement path when (a) the C-arm 14 is stopped for the predetermined time or longer, (b) X-rays are radiated, or (c) the C-arm 14 moves for the predetermined time or longer at the designated speed or slower. The display control function 746 causes the display 72 to further display the above-described specific positions on the moving image.

It is conceivable that the user moves the C-arm 14 in a relatively slower manner at the time of imaging important region. Thus, the position of the C-arm 14 at the time of imaging the above-described important region can be stored by storing the position when the C-arm 14 moves at the designated speed or slower for a predetermined time or longer. This configuration enables to store the position of the movement of the C-arm 14 that the user is more likely to reproduce.

Note that the storage control function 745 may store the final position after moving the C-arm 14 at a speed equal to or slower than the designated value. In addition, the storage control function 745 may also store the range in which the C-arm 14 is moved at a speed equal to or slower than the designated value. At this time, the storage control function 745 may store the start position at a time point at which the speed becomes a predetermined value or slower.

### Fourth Embodiment

Although a description has been given of the case where the imaging apparatus is the C-arm 14 in the first to third embodiments, the first to the third embodiments are also applicable to the X-ray CT apparatus 30 and the bed apparatus 50. In other words, when the X-ray CT apparatus 30 and/or the table 53 are moved in combination with the C-arm 14, the position storage processing and the reproduction-and-storage processing are performed in a similar manner.

FIG. 11 is a block diagram illustrating a configuration of the X-ray diagnostic apparatus 1a according to the fourth embodiment. FIG. 12A and FIG. 12B are perspective views illustrating the appearance of the X-ray diagnostic apparatus 1a. The X-ray diagnostic apparatus 1a includes the X-ray angiography apparatus 10, the X-ray CT apparatus 30, the bed apparatus 50, and the console 70. The components other than the X-ray CT apparatus 30 are the same as those in the first embodiment.

The X-ray CT apparatus 30 includes an X-ray tube 31, an X-ray detector 32, a rotating frame 33, an X-ray high-voltage generator 34, the CT controller 35, a wedge 36, a collimator 37, and a DAS 38. Although a plurality of X-ray CT apparatuses 30 are illustrated in FIG. 11 for showing the relationship with the bed apparatus 50, the actual number of the X-ray CT apparatus 30 in this embodiment is only one.

The X-ray tube 31 generates X-rays. Specifically, the X-ray tube 31 is a vacuum tube that holds a cathode configured to generate thermoelectrons and an anode configured to receive thermoelectrons from the cathode and thereby generate X-rays. The X-ray tube 31 is connected to the X-ray high-voltage generator 34 via a high-voltage cable. A tube voltage is applied between the cathode and the anode by the X-ray high-voltage generator 34. When a tube voltage is applied, the thermoelectrons move from the cathode to the anode, and thereby, a tube current flows. Supply of a filament current and application of a high voltage from the X-ray high-voltage generator 34 causes the thermoelectrons to move from the cathode to the anode, and X-rays are generated by having the thermoelectrons collide with the anode.

The X-ray detector 32 detects the X-rays having been emitted from the X-ray tube 31 and having passed through the object P, and outputs an electrical signal corresponding to the X-ray dose to the DAS 38. The X-ray detector 32 has a plurality of X-ray detection element rows, in each of which a plurality of X-ray detection elements are arranged in the channel direction along a circular arc centered on the focal point of the X-ray tube, for example. The X-ray detector 32 has a structure in which the plurality of X-ray detection element rows are arranged in the slice direction (i.e., row direction), and a plurality of X-ray detection elements are arranged in the channel direction in each X-ray detection element row, for example. In addition, the X-ray detector 32 is an indirect conversion type detector having a grid, a scintillator array, and an optical sensor array, for example.

The scintillator array has a plurality of scintillators, and each scintillator has a scintillator crystal configured to output light of photons in an amount corresponding to incident X-ray dose.

The grid is disposed on the surface of the X-ray incident side of the scintillator array, and has an X-ray shielding plate that has a function of absorbing scattered X-rays. Note that the grid is also called a collimator (one-dimensional collimator or two-dimensional collimator).

The optical sensor array has a function of converting the light from the scintillator into an electrical signal corresponding to the amount of light, and includes an optical sensor such as a photomultiplier tube (PMT).

The X-ray detector 32 may be a direct conversion type detector that has a semiconductor element configured to convert incident X-rays into electrical signals.

The rotating frame 33 is an annular frame that rotatably supports the X-ray tube 31 and the X-ray detector 32 around an axis in the Z direction. Specifically, the rotating frame 33 supports the X-ray detector 32 and the X-ray tube 31, both of which are housed in the X-ray CT apparatus 30 and are arranged so as to face each other through an aperture 30a. The rotating frame 33 is rotatably supported by a fixed frame (not shown) around the axis in the Z direction. The CT controller 35 rotates the X-ray tube 31 and the X-ray detector 32 around the axis in the Z direction by rotating the rotating frame 33 around the axis in the Z direction. The rotating frame 33 receives power from a drive mechanism of the CT controller 35 so as to rotate at a constant angular velocity around the axis in the Z direction. An image field of view (FOV) is set in the aperture of the rotating frame 33.

The X-ray high-voltage generator 34 includes: a high voltage generator that has electric circuits, such as a transformer and a rectifier, and generates the high voltage to be applied to the X-ray tube 31 and the filament current to be supplied to the X-ray tube 31; and an X-ray controller configured to control the output voltage corresponding to the X-rays to be emitted by the X-ray tube 31. The high voltage generator may be of a transformer type or an inverter type. The X-ray high-voltage generator 34 may be provided on the rotating frame 33 in the X-ray CT apparatus 30 or may be provided on the fixed frame (not shown) in the X-ray CT apparatus 30.

The CT controller 35 controls the X-ray high-voltage generator 34 and the DAS 38 in accordance with an imaging control function 733 of the processing circuitry 74 of the console 70 in order to perform X-ray CT imaging. The CT controller 35 includes: processing circuitry having components such as a CPU; and a drive mechanism such as a motor and an actuator. The processing circuitry has a processor such as a CPU and an MPU and a memory such as a ROM and a RAM, as hardware resources. The CT controller 35 may also be realized by an ASIC, an FPGA, a CPLD, or a SPLD.

The CT controller 35 has a function to control the respective operations of the X-ray CT apparatus 30 and the bed apparatus 50 in response to an input signal from the input interface 73, which is attached to the console 70 or the X-ray CT apparatus 30 and is described below. For example, the CT controller 35 performs: control of rotating the rotating frame 33 in response to the input signal; control of tilting the X-ray CT apparatus 30; and control of operating and moving the bed apparatus 50 and the table 53. The control of tilting the X-ray CT apparatus 30 is achieved by causing the CT controller 35 to rotate the rotating frame 33 around an axis parallel to the X direction on the basis of tilt angle information inputted via the input interface 73 that is attached to the X-ray CT apparatus 30. Note that the CT controller 35 may be provided in the X-ray CT apparatus 30 or in the console 70.

The wedge 36 adjusts the dose of X-rays to be radiated to the object P. Specifically, the wedge 36 attenuates the X-rays in such a manner that the dose of X-rays to be radiated from the X-ray tube 31 to the object P has a predetermined distribution. For example, a metal plate (e.g., aluminum) such as a wedge filter and a bow-tie filter is used as the wedge 36.

The collimator 37 limits the irradiation range of the X-rays that have passed through the wedge 36. The collimator 37 slidably supports a plurality of lead plates that shield X-rays, and adjusts the form of slits formed by the plurality of lead plates.

The DAS 38 receives information on the incident channel outputted from the X-ray detector 32 for each view period, and thereby acquires detection data having a digital value corresponding to the X-ray dose over the view period. The DAS 38 is achieved by an ASIC that is provided with circuit elements capable of generating detection data, for example. The detection data are transmitted to the console 70 via a non-contact data transmission device, for example.

The X-ray CT apparatus 30 includes various types such as: a rotate/rotate type (i.e., third generation CT) in which the X-ray generator and the X-ray detector integrally rotate around an object; and a stationary/rotate type (i.e., fourth generation CT) in which a large number of X-ray detection elements arrayed in a ring shape are fixed and only the X-ray generator rotates around the object, and any type can be applied to the embodiment.

In FIG. 12A, the C-arm 14 is close to the table 53, whereas the X-ray CT apparatus 30 is away from the table 53. In FIG. 12B, the C-arm 14 is located further away from the table 53, whereas the X-ray CT apparatus 30 is closer to the table 53.

Specifically, as shown in FIG. 12A and FIG. 12B, the C-arm 14 is capable of the parallel movement under a plurality of rails r2 provided on the ceiling along the long axis direction or the short axis direction of the table 53. In addition, the C-arm 14 is capable of the parallel movement along the rails r2, along the long axis direction and the short axis direction of the table 53 by a moving mechanism (not shown). Further, the C-arm 14 is supported by the support arm 14b via a holding portion 14a. The support arm 14b has a substantially arcuate shape, and its base end is attached to a moving mechanism for moving along and under the rails r2.

When the X-ray CT apparatus 30 and the table 53 are moved together with the C-arm 14, the memory 71 corresponding to the left part of FIG. 5 stores the movement position of the X-ray CT apparatus 30 and the angle of the table 53 together with the angle and movement position of the C-arm 14 at predetermined time intervals. In other words, the movements of the plurality of movable shafts of the imaging apparatus are stored for each time period between two adjacent time axes.

According to at least one embodiment described above, the movement path of the imaging apparatus can be reproduced in the X-ray diagnostic apparatus.

While certain embodiments have been described, these embodiments have been presented by way of example only, and are not intended to limit the scope of the invention. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
a memory; and
processing circuitry configured to
cause the memory to store a movement path that is defined by a combination of movements of a plurality of movable shafts of an imaging apparatus and is generated based on movements of the imaging apparatus in response to a user's operation and
move the imaging apparatus in a reverse order of the movement path stored in the memory.

2. The X-ray diagnostic apparatus according to claim 1, wherein the processing circuitry is configured to move the imaging apparatus in the reverse order when an instruction to move the imaging apparatus in the reverse order is continued by the user's operation.

3. The X-ray diagnostic apparatus according to claim 1, wherein the processing circuitry is configured to cause the memory to store the movement path generated based on movements of the imaging apparatus in response to a manual operation in which the user manually sets the plurality of movable shafts.

4. The X-ray diagnostic apparatus according to claim 1, wherein the processing circuitry is configured to cause the memory to store the movement path generated based on movements of the imaging apparatus in response to the user's operation performed between a plurality of movements of the imaging apparatus in accordance with an auto-positioning instruction.

5. The X-ray diagnostic apparatus according to claim 1, wherein the processing circuitry is configured to cause the memory to store the movement path generated based on movements of the imaging apparatus in response to the user's operation related to auto-positioning that reads out and executes a pre-registered motion setting of the movable shafts.

6. The X-ray diagnostic apparatus according to claim 1, wherein the plurality of movable shafts of the imaging apparatus include a plurality of movable shafts of a C-arm of an X-ray angiography apparatus and a plurality of movable shafts of a bed.

7. The X-ray diagnostic apparatus according to claim 6, wherein the plurality of movable shafts of the imaging apparatus further include a movement shaft of the X-ray CT apparatus with respect to the bed, the X-ray CT apparatus being moved in combination with the X-ray angiography apparatus.

8. The X-ray diagnostic apparatus according to claim 1, wherein the processing circuitry is configured to move the imaging apparatus in such a manner that the forward-direction movement path is reproduced in a reverse direction.

9. The X-ray diagnostic apparatus according to claim 1, wherein the processing circuitry is configured to cause the memory to store a combination of movements of the plurality of movable shafts of the imaging apparatus at predetermined time intervals as the movement path.

10. The X-ray diagnostic apparatus according to claim 1, wherein the processing circuitry is configured to:
receive, as a stop position of the imaging apparatus, one position that is on the stored movement path of the imaging apparatus and is a position designated by the user; and
automatically move the imaging apparatus from an arbitrary position on the movement path to the stop position in response to a start instruction from the user.

11. The X-ray diagnostic apparatus according to claim 1, further comprising a display,
wherein the processing circuitry is configured to cause the display to display a pseudo image of the imaging apparatus corresponding to each position on the stored movement path as a moving image.

12. The X-ray diagnostic apparatus according to claim 11, wherein the processing circuitry is configured to:
cause the display to further display a seek bar that allows the user to designate a position corresponding to an arbitrary frame of the moving image as a stop position of the imaging apparatus; and
move the imaging apparatus until the imaging apparatus reaches the stop position.

13. The X-ray diagnostic apparatus according to claim 11, wherein the processing circuitry is configured to:
cause the memory to further store a specific position on the movement path when (a) the imaging apparatus is stopped for a predetermined time or longer, (b) X-rays are radiated, or (c) the imaging apparatus moves for a predetermined time or longer at a designated speed or slower; and
cause the display to further display the specific position on the moving image.

14. The X-ray diagnostic apparatus according to claim 1, wherein:
if the movement path is defined as a first path, when the imaging apparatus is moving in a reverse order of the first path, the processing circuitry causes the memory to further store the movement path in the reverse order as the second path; and
the processing circuitry can further move the imaging apparatus in a reverse order of the second path.

15. The X-ray diagnostic apparatus according to claim 1, wherein the processing circuitry is configured to, in accordance with a user's selection, move the imaging apparatus from a current position to a target position along a shortest path instead of the movement path stored in the memory.

16. A control method for an X-ray diagnostic apparatus comprising steps of:
causing a memory to store a movement path that is defined by a combination of movements of a plurality of movable shafts of an imaging apparatus and is generated based on movements of the imaging apparatus in response to a user's operation; and
moving the imaging apparatus in a reverse order of the movement path stored in the memory.
